(19) United States
(12) Patent Application Publication
(10) Pub. No.: US 2023/0215533 A1
(43) Pub. Date: Jul. 6, 2023

(54) AN ONLINE HEALTH-RELATED SERVICES PLATFORM AND A METHOD PROVIDING THEREOF

(71) Applicant: DNA UP COMMUNICATION LTD, GEULIM (IL)

(72) Inventor: HAIM OHAYON, GEULIM (IL)

(21) Appl. No.: 18/008,553

(22) PCT Filed: Jun. 9, 2021
(86) PCT No.: PCT/IL2021/050692

§ 371 Date: Dec. 6, 2022

(30) Foreign Application Priority Data
Jun. 11, 2020 (IL) 275320

(51) Int. Cl. G16H 20/00; G16H 50/70

(52) U.S. Cl. CPC G16H 20/00; G16H 50/70

(57) ABSTRACT

A method for providing an online digital medical & health-related services platform based on personal experience for pregnant and postnatal women and families, for personally customizing different services depending on the pregnancy stage or the infant age, their needs, preferences, interests, living environment/conditions, lifestyle, health status, etc. According to an embodiment of the invention, the proposed platform offers expedited enrollment/registration to relevant services, such as a medical newborn insurance policy. The proposed platform provides a short enrollment, approval, and activation of the insurance policy in an expedited enrollment process.

### FIELD OF THE INVENTION

The present invention relates to the field of digital platforms. More particularly, the invention relates to an online digital medical & health-related services platform based on personal experience for pregnant and postnatal women and young families.

### BACKGROUND OF THE INVENTION

Pregnant and postnatal women can adapt various services suiting to the stage of pregnancy and fetal development and/or postpartum infant age, according to the interests and preferences of each woman, her age, her lifestyle and her family's lifestyle, sources of residence, living environment, previous purchases she has made and more. Examples of the various services that can be offered to pregnant and/or postpartum women: obstetrician or pediatrician counseling, sports activities such as Yoga, Pilates, pregnant women's classes, childbirth preparation courses, postpartum counseling courses such as sleep counseling, breastfeeding counseling, pregnancy products such as maternity clothes, pregnancy cosmetics, purchase products for mother and baby before and/or after birth, etc.

In the conventional mother care service, the mother or the guardian has to go directly to the mother-related service provider such as the obstetrician, the maternity care center, and the maternity/babies shop to consult the service and/or purchase products, and to receive the reservation. In such a case, it is inconvenient for mothers who have inconvenient mobility to go directly to the service provider to consult and make a reservation.

In addition, there is a problem that it is almost inefficient to find a service suitable for the mother by selling the product directly to the mother at a time when the number of maternity-related service providers is increasing. In general, mothers are exposed to maternal services through internet cafes, blogs, and search sites. They cannot consider their lifestyle, living environment, and health status in a comprehensive manner. Therefore, in order to make the service giving more efficient, there is a need for suiting services personally to each pregnant or postnatal woman, according to different criteria that fit specifically to the woman and according to the pregnancy stage or the infant age.

It is, therefore, an object of the present invention to provide a platform based on personal experience for pregnant and postnatal women and families, for personally customizing different services depending on the pregnancy stage or the infant age, their needs, preferences, interests, living environment/conditions, lifestyle, health status, etc.

Other objects and advantages of the invention will become apparent as the description proceeds.

### SUMMARY OF THE INVENTION

A method for personally customizing services for pregnant and postnatal women and young families, comprising:
collecting data relative to each specific user;
processing said collected data according to different pregnancy stages or infant's age for postnatal women and accordingly building a user unique “persona” profile, wherein said “persona” profile represents a character from users' target audience;
comparing said user unique “persona” profile with previously similar built “persona” profiles of other users;
providing service(s) proposals according to said unique “persona” profile; and
enriching said collected data according to said previously similar built “persona” profiles of other users and accordingly updating each specific “persona” profile.

According to an embodiment of the invention, the collected data obtained from one or more sources, location services and data input from users.

According to an embodiment of the invention, the one or more sources are selected from the group consisting of: medical database(s), online sources, social networks, data collected from mobile devices, or any combination thereof.

According to an embodiment of the invention, the service(s) proposals are provided by third parties, including places where one can buy goods or services, either online or traditional places, medical services, health and fitness services, etc.

According to an embodiment of the invention, the service(s) are expedited enrollment to one or more newborn medical insurance policies, and wherein the collected data includes newborn's demographical and medical details that are ustilized as actuarial data for performing risk assessment and for generating corresponding insurance policy quotations.

In another aspect, the present invention relates to a system for personally customizing services for pregnant and postnatal women and young families, comprising:
at least one processor; and
a memory comprising computer-readable instructions which when executed by the at least one processor causes the processor to execute an online digital medical & health-related services platform configured for providing personally customizing services for pregnant and postnatal women and young families, wherein the platform:
   i. collects data relative to each specific user;
   ii. processes said collected data according to different pregnancy stages or infant's age for postnatal women and accordingly building a user unique “persona” profile, wherein said “persona” profile represents a character from users' target audience;
   iii. compares said user unique “persona” profile with previously similar built “persona” profiles of other users;
   iv. provides service(s) proposals according to said unique “persona” profile; and
   v. enriches said collected data according to said previously similar built “persona” profiles of other users and accordingly updating each specific “persona” profile.

According to an embodiment of the invention, the system further comprises enabling users to interact with said platform via a website and/or an application.

According to an embodiment of the invention, the system further comprises a social network for allowing pregnant/postnatal women to have personal conversations on maternity with other pregnancies, fathers, and young mothers.

According to an embodiment of the invention, the system further comprises allowing the integration of ONLINE and OFFLINE management of the various stages of pregnancy and infants' development in the platform via a Customer Relationship Management (CRM) service/system.

According to an embodiment of the invention, personal conversations of pregnant/postnatal women with other pregnancies, fathers and young mothers in the social network can be utilized for creating friendships, giving and receiving tips and viral information, content transfer, etc., and also to make recommendations for the platform system, such as reviews of missing products or service gap encountered by the user which would like to receive a response from the community about this gap.

### BRIEF DESCRIPTION OF THE DRAWINGS

The above and other characteristics and advantages of the invention will be better understood through the following illustrative and non-limitative detailed description of preferred embodiments thereof, with reference to the appended drawings, wherein:

FIG. 1 shows a block diagram of the proposed platform's parts and components, according to an embodiment of the present invention;

FIG. 2 shows a flow chart of the proposed platform's method steps (stages), according to an embodiment of the present invention;

FIG. 3 shows a flow chart of a medical newborn insurance policy enrollment process, according to an embodiment of the present invention; and

FIG. 4 shows an exemplary screen layout that is part of an insurance policy expedited enrollment process, according to an embodiment of the present invention.

### DETAILED DESCRIPTION OF THE INVENTION

The present invention provides an online medical & health-related services platform based on personal experience for pregnant and postnatal women and young families.

The proposed platform (Health Tech) combines different areas of consumerism. Inside the platform, a virtual mall (Market Place) of leading brand stores is established, while the platform operation is related to an algorithm that personally identifies the users' needs and suits their products and services from the virtual mall that suit each specific user's needs. These products and services are proposed to the users by the platform. The users can also search by categories of different services/products on the platform.

The present invention's method utilizes various tools to perform smart personal audience “painting” of pregnant and postpartum women. The tools are based on developed code and algorithms in addition to physical data collected at many medical centers and unique promotion activities. The whole tools feed each other on a big data platform to accurately identify audiences of pregnant and postpartum women and young families who need/will be interested in different services.

Once the data for a specific user is collected, continuous parameters are analyzed to make unique services suggestions that are personally suit-made for the pregnancy stage (pregnancy week) or the age of the infant, in another consideration of customer residence, interests, purchases from different sites, etc., producing hundreds and thousands of personal users' “journeys” simultaneously a day. The terms “journey” or “digital journey” refers herein to an automatic process of sending different offers to users that were built as part of the characterization of each user's persona. All the offers together create one journey. For example, a referral to an article and then a referral to watch a video, and then an offer to purchase a product that was mentioned or appeared in the article and/or video. The system may automatically send different offers to a user at different timings (i.e., such as push notifications).

Within the Market Place, the ability to access personal journeys organically within the website browsing experience has been developed uniquely for each user by changing messages to maximize propulsion for action. In other words, a user can access a personal area in the digital marketplace and view all the available offers for him.

In addition, a unique personalized domain is developed by the present invention's method for each user, where all the content from all the domains that have been marked as favorites by the user will appear, and thus a completely personal experience will be built for the user (a personal domain that a user builds for herself/himself from contents and platform's values propositions). In accordance with the same preferences, additional user-customized contents will also be accessed to the user, based on data management from combinations and prediction models, according to similar user's preferences and purchases.

The present invention creates a young families community-a unique closed social network which has been developed in the Market Place community for young families that allows pregnant/postnatal women to have personal conversations with other pregnancies as to maternity and/or fathers and/or young mothers, etc., for creating friendships, giving and receiving tips and viral information, content transfer, and more, and also to make recommendations for the platform system, such as reviews of missing products or service gap encountered by the user which would like to receive a response from the community about this gap. For example, an offer for a specific unsold product, an offer for insurance services for families who find it difficult to obtain an attractive price, etc.

In addition, in order to encourage the user to share the platform with her/his acquaintances and her/his family, a wish list was developed by the present invention, enabling the user to create/prepare various lists of products or services and pass them through digital tools to various groups, including the user's private instant messaging service (e.g., WhatsApp messenger or other cross-platform centralized messaging and voice-over-IP (VoIP) service), or any other distribution channel. For example, a user shares her/him pre-delivery purchases between friends, family, work members, etc.

The Website

The proposed website can be based on a combination of an open code developed in WordPress or a general-purpose scripting language especially suited to web development (e.g., PHP) and an algorithm that may provide personal prediction (e.g., that can be developed in Payton and embedded in the platform). This is combined with unique components such as the digital journey, user's wish list, etc., of the present invention's method.

The website is based on a social network that accompanies the mother from the initial stages of pregnancy throughout and after the birth process. Aspects of emotional support accompany the website (a list of expert consultants with whom the user can chat, video-chat and coordinate a meeting diary online, without any human contact). In The Market Place, there is also escorting and purchasing of products, based on many stores within collaborations in various frameworks.

The sign-up process to the website can be initialized in multiple processes, with flexible adaptation to the source of the process and a personally customized view (display) for each user.

The website allows online domain management for medical centers (Health Tech) in a flexible and unique manner to any medical center. The medical domains allow each domain administrator to manage the content independently by means of a permissions mechanism. The medical complex enables management of conversations in chat, video online, and coordinating a meeting with a diary accessible to the medical center's experts, everything online without human contact, including live broadcasts, one-to-one workshops, group workshops, and scheduling appointments based on a personal diary, for a variety of service providers. The website also incorporates components that allow group and one-to-one calls in addition to live-event broadcasts.

The insurance domain includes a dedicated domain for young families, studying by the algorithm abilities the needs of young families according to personal data, fits them policies personally, and predicts which clients are more likely to convert to a policy in a statistically higher way (DATA-Based Insurance).

For convenience, the site interfaces with a CRM system and allows the integration of ONLINE and OFFLINE management of the various stages of pregnancy and infants' development. The website may include dynamic components that enable dynamic and flexible collaboration with third parties and external providers. For example, when a user logs in to the website, some of the site components (such as special offers, commercials, information segments, etc.) may change according to what the system concluded is the best fit for this specific user according to the collected data and learning (i.e., by using an application or other software module(s) of artificial intelligence (AI) that provides the system of the present invention the ability to automatically learn and improve from experience without being explicitly programmed).

In addition, the platform includes buying/selling second-hand products and indexing jobs suitable for young mothers.

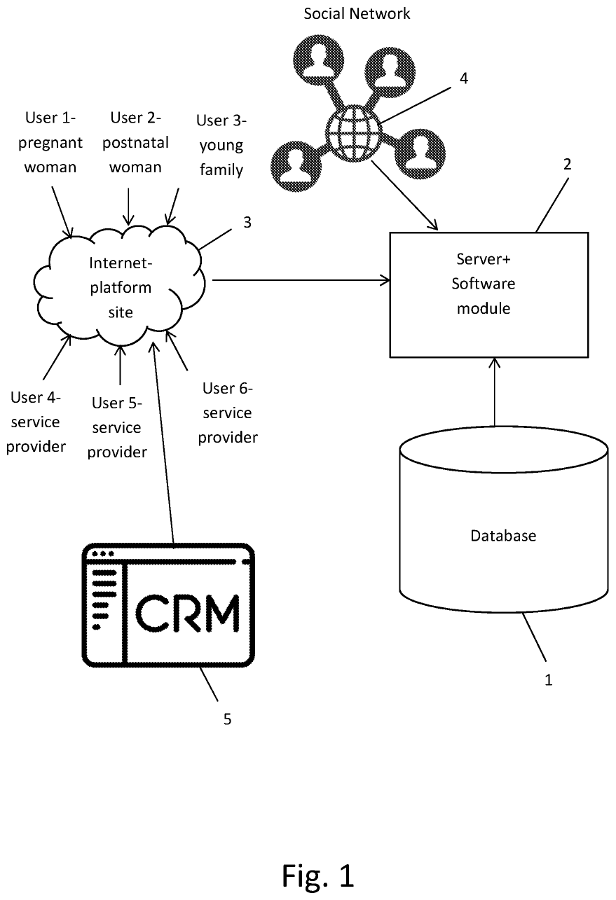

FIG. 1 shows a block diagram of the proposed platform's parts and components, according to an embodiment of the present invention. Database 1 contains data on pregnant and postnatal women and young families, and is connected to a server with software module 2, which includes the developed algorithm and codes of the platform. Server 2 is connected to website 3 through the Internet, in which all the users (can be pregnant women, postnatal women and young families, or service providers like sellers, shops, doctors, therapists, medical centers, etc.) register to the platform, and their details are entered to the database 1. Server 2 is also connected to social network 4 for allowing pregnant/postnatal women to have personal conversations with other pregnancies/postnatal women as to maternity and/or fathers and/or young mothers, etc., for creating friendships, giving and receiving tips and viral information, content transfer, etc., and also to make recommendations for the platform system such as reviews of missing products or service gap encountered by the user which would like to receive a response from the community about this gap. Server 2 is also connected to CRM system 5, for allowing the integration of ONLINE and OFFLINE management of the various stages of pregnancy and infants' development in the platform.

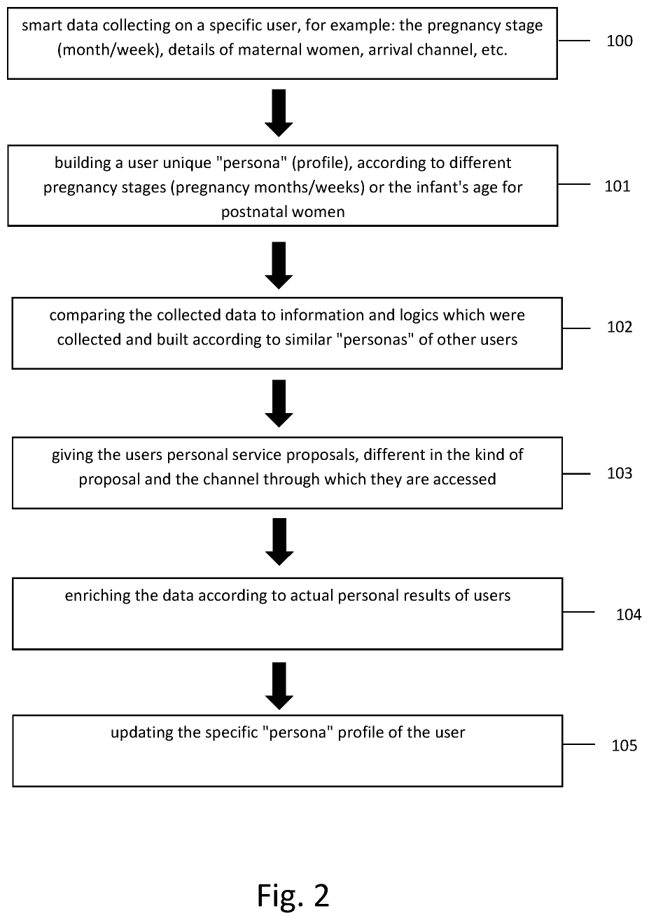

FIG. 2 shows a flow chart of the proposed platform's method steps (stages), according to an embodiment of the present invention. At the first step 100, smart data collecting on a specific user, for example: the pregnancy stage (month/week), details of pregnant/postnatal women, arrival channel (i.e., the channel represents the action that led to the user's first engagement with the suggested platform. For example, a publish on Facebook, registration to an event for pregnant women, signing up for a tour at the hospital's maternity ward and the like), etc. At the next step 101, building a user unique “persona” (a user profile, a character that represents a part from the target audience), according to different pregnancy stages (months/weeks) or the infant's age for postnatal women. At the next step 102, comparing the collected data to information and logics which were collected and built according to similar “personas” of other users. At the next step 103, giving the user personal service proposals, different in the kind of proposal and the channel through which they are accessed. At the next step 104, enriching the data according to actual personal results of users. At the next step 105, updating the specific “persona” profile of the user.

The Uniqueness of the Proposed Platform

The platform proposed by the present invention employs various tools to “paint” smart audiences of pregnant and postnatal women and young families. The tools are based on digital capabilities in addition to physical data collected at many medical centers and unique promotional activities. All tools feed one another to accurately identify audiences.

The digital “personas” of the different users which are built by the proposed platform are different from prior-art “personas” managing because the management and characterization are focused on the different stages of pregnancy (pregnancy month/week) and the infant's age for postnatal women. The information is collected and divided according to criteria tested on over 200,000 different women in accordance with the accuracy of the specific pregnancy week.

Beyond the pregnancy stage, the proposed platform model also refers to data such as the user's place of residence, expected birth place, previously consumed content by the user, services/products previously purchased by the user, how the user joined the database, etc., and uses all the data to precisely the “persona” creation.

The proposed platform is accessible to the users through various tools, most notably the Market Place, which are personally suit-made for the model built for the user and evolve continuously according to the progress of pregnancy and information collection. Any activity that the user begins will actually take place in the Market Place, enabling the behavior analysis to be deepened and making the user's personal model more accurate (which contents have been viewed by the user, for how long, which pages the user examined, and more).

Every action that the user takes (or chooses not to take) in the Market Place is automatically updated to the personal database of the platform, and the data is aggregated anonymously to the forecasting model in order to continue to accurate the various users'“personas” and actions.

According to an embodiment of the invention, the proposed platform may be utilized to offer expedited enrollment/registration to relevant services, such as a medical newborn insurance policy, where the proposed platform provides a short enrollment, approval, and activation of the insurance policy in an expedited enrollment process (e.g., which may be completed by up to one minute).

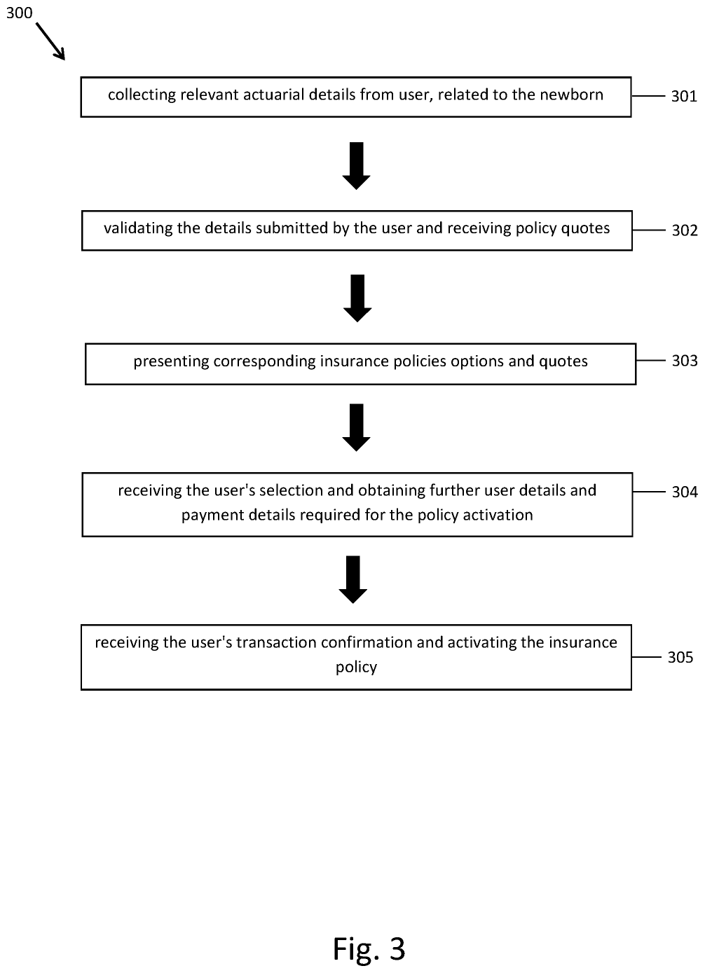

FIG. 3 shows a flow chart of a medical newborn insurance policy enrollment process 300 steps (stages), according to an embodiment of the present invention. In step 301, the proposed platform collects relevant identifying information from a user (e.g., mother, father, etc.) related to the newborn, such as demographic details (e.g., the newborn's gender), and general medical details (e.g., further medical consultations/procedures required). Such information may provide actuarial data based on which suitable insurance policy rates can be generated.

In step 302, server 2 (of FIG. 1), utilizes database 1, social network 4, and/or further information resources (e.g., one or more insurance provider's computers), for validating the details submitted by the user, such as to validate his/hers identity and for receiving corresponding insurance policy quotes. Of course, the proposed platform may present to the user relevant privacy and/or medical disclaimers for receiving confirmation to collect the required information from authorization requiring data sources. In step 303, based on the insurance policy quotes received from one or more insurance policies providers, insurance policies options are presented to the user, and in steps 304 and 305, the user's selection and complementary required personal and payment details are received and the selected insurance policy(ies) is activated at the corresponding insurance policy provider(s).

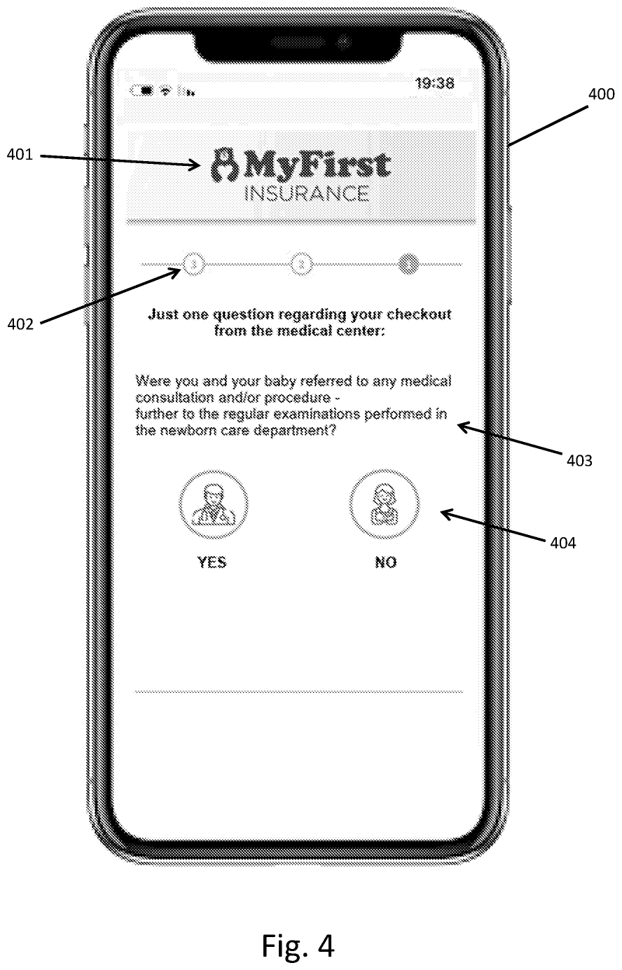

FIG. 4 shows an exemplary screen layout of step 302 by a mobile device 400, according to an embodiment of the present invention. While multiple different website/mobile application designs may be selected, by one skilled in the art, for use in conjunction with the proposed platform, a common screen display of step 302 may include a logo 401 of the service provider and/or sponsoring insurance provider, an enrollment progress ruler 402 showing the current enrollment stage with respect to the overall enrollment, a question to the enrolling user 403 asking whether further medical consultations/procedures are required (i.e., such information can be used for performing actuary risk assessment and for generating corresponding policy price quotes), and user selection operators, such as YES/NO touch operators for entering the user's selection.

All the above descriptions and examples have been given for the purpose of illustration and are not intended to limit the invention in any way. Many different methods of data processing and services can be employed, all without exceeding the scope of the invention.

1. A method for personally customizing services for pregnant and postnatal women and young families, comprising:
a) collecting data relative to each specific user;
b) processing said collected data according to different pregnancy stages or infant's age for postnatal women and accordingly building a user unique “persona” profile, wherein said “persona” profile represents a character from users' target audience;
c) comparing said user unique “persona” profile with previously similar built “persona” profiles of other users;
d) providing service(s) proposals according to said unique “persona” profile; and
e) enriching said collected data according to said previously similar built “persona” profiles of other users and accordingly updating each specific “persona” profile.

2. The method according to claim 1, wherein the collected data is obtained from one or more sources, location services, and data input from users.

3. The method according to claim 2, wherein the one or more sources are selected from the group consisting of: medical database(s), online sources, social networks, data collected from mobile devices, and any combination thereof.

4. The method according to claim 1, wherein the service(s) proposals are provided by third parties that provide goods or services, either online or traditional places.

5. The method according to claim 1, wherein the service(s) are expedited enrollment to one or more newborn medical insurance policies, and wherein the collected data includes newborn's demographical and medical details that are utilized as actuarial data for performing risk assessment and for generating corresponding insurance policy quotations.

6. A system for personally customizing services for pregnant and postnatal women and young families, comprising:
at least one processor; and
a memory comprising computer-readable instructions which, when executed by the at least one processor, causes the processor to execute an online medical & health-related services platform configured for providing personally customizing services for pregnant and postnatal women and young families, wherein the platform:
i) collects data relative to each specific user;
ii) processes said collected data according to different pregnancy stages or infant's age for postnatal women and accordingly building a user unique “persona” profile, wherein said “persona” profile represents a character from users' target audience;
iii) compares said user unique “persona” profile with previously similar built “persona” profiles of other users;
iv) provides service(s) proposals according to said unique “persona” profile; and
v) enriches said collected data according to said previously similar built “persona” profiles of other users and accordingly updates each specific “persona” profile.

7. The system according to claim 6, further comprising means for enabling users to interact with said platform via a website and/or an application.

8. The system according to claim 6, further comprising a social network for allowing pregnant/postnatal women to have personal conversations on maternity with other pregnant women, fathers, and young mothers.

9. The system according to claim 6, further comprising means for allowing the integration of ONLINE and OFFLINE management of the various stages of pregnancy and infants' development in the platform via a Customer Relationship Management (CRM) service/system.

10. The method according to claim 4, wherein the services provided by third parties are selected from medical services, and health and fitness services.
